(19) United States
(12) Patent Application Publication
(10) Pub. No.: US 2007/0231377 A1
(43) Pub. Date: Oct. 4, 2007

(54) COMPOSITIONS FOR PROMOTING HAIR GROWTH

(75) Inventor: Ibrahim Abou-Nemeh, Lake St. Louis, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: Novus International Inc., St. Louis, MO (US)

(21) Appl. No.: 11/692,569

(22) Filed: Mar. 28, 2007

Related U.S. Application Data
(60) Provisional application No. 60/786,754, filed on Mar. 28, 2006.

(51) Int. Cl. A61K 31/19; A61K 9/127

(52) U.S. Cl. 424/450; 514/557

(57) ABSTRACT

The present invention provides compositions and methods for treating hair loss, nail brittleness and skin conditions, and for promoting or enhancing hair growth. The composition generally includes at least one methionine analog or derivative. The invention further provides structured fluid delivery systems comprising the compositions of the invention.

### CROSS-REFERENCE TO RELATED APPLICATION

This application claims priority to U.S. Provisional Application Ser. No. 60/786,754 filed on Mar. 28, 2006, which is hereby incorporated by reference in its entirety.

### FIELD OF THE INVENTION

The present invention provides compositions and methods for promoting hair growth, and for treating nail brittleness and skin conditions. The composition generally includes at least one methionine analog or derivative.

### BACKGROUND OF THE INVENTION

Hair loss and baldness are a common affliction in mammals. Over forty percent of the male and nearly fifteen percent of the female human population suffers from some form of hair loss or baldness. It is only in recent years, with a greater knowledge of genetics and the chemistry of sexual hormones, that some of the causes of hair loss have been elucidated. While hair loss may be induced by a variety of transient conditions such as chemotherapy treatment, fever, or diet, the most prevalent factors causing hair loss in humans is a combination of genetics and hormones. In men, this type of hair loss, also called male pattern baldness or adrogenetic alopecia, is responsible for more than 95% of all hair loss. The precise cause of androgenetic alopecia is not known. Although it is clear that in addition to genetics, the level of androgens (e.g., testosterone and dihydrotestosterone) plays a significant role. Simply put, the more androgens an individual has, the faster they lose their hair.

In androgenetic alopecia, hair follicles that are producing healthy, terminal hairs begin to produce thinner, shorter, more brittle hairs with weaker shafts. Eventually, these follicles produce only fine, almost invisible, short, vellus hairs, or they may die out altogether. It is these hair follicles that cause the common hair loss pattern: first receding temples, then thinning on the top, followed by a bald spot on the crown, and eventually no hair at all on the top of the head. Usually, hair on the sides and on the back of the head remains, even with advanced inherited pattern hair loss.

Irrespective of the cause underlying hair loss or baldness, one thing is know for certain: no matter what humans eat, what their lifestyles may be, or what kind of vitamins they take, they never grow more hair follicles than the approximately 150,000 they were born with. Hair follicles grow hairs. When the ones with a genetic program to stop growing reach a certain age, they begin to produce hairs that are thinner, shorter, and lighter. Eventually they just stop producing new hairs altogether. When enough hair follicles on one part of our heads do this, a thin spot appears. And, when they all stop producing, baldness results.

Because new hair follicles cannot presently be regenerated, treatment strategies focus on preserving them. Common treatment strategies include topical treatments, medicinal treatments, and hair restoration surgery. Topical and oral treatment with drugs, such as minoxidil (i.e., sold under the trade name Rogain® by the UpJohn Company previously, and a Pfizer Inc. recently) or finasteride (i.e., sold under the trade name Propecia® by Merck and Company), are effective for treating androgenetic alopecia in some, but not all individuals. While effective, both drugs have significant drawbacks. Minoxidil, in addition to being cost prohibitive for a significant segment of the population, is a vasodilatory drug that can cause change in heart rate or blood pressure and chest pain-if excessive amounts of it are absorbed through the skin. In males, propecia can cause sexual dysfunction, breast tenderness and breast enlargement, and possible prostate enlargement. Although hair restoration surgery may be effective, it is excruciatingly painful and cost prohibitive. A need, therefore, exists for compositions that are effective at stimulating hair growth in a cost efficient manner and with few negative side effects.

### SUMMARY OF THE INVENTION

One aspect of the invention encompasses a method for promoting hair growth in a subject. The method comprises administering a hydroxy analog of methionine and its chemical and physical derivatives to the subject.

Yet another aspect of the invention provides a composition that is effective for treating alopecia and for promoting hair growth in a subject. The composition comprises a structured fluid delivery system containing a hydroxy analog of methionine and its chemical and physical derivatives.

An additional aspect of the invention encompasses a method for selectively delivery a hydroxy analog of methionine to a skin cell or hair follicle of a subject. The method comprising topically applying to the skin of the subject a composition having a structured fluid delivery system containing a hydroxy analog of methionine. Typically, the structured fluid delivery system selectively delivers the hydroxy analog of methionine to a hair follicle or skin cell disposed within the subject's skin.

Other aspects and features of the invention are described in more detail herein.

### DETAILED DESCRIPTION OF THE PREFERRED EMBODIMENTS

It has been discovered that certain methionine compounds are effective for treating alopecia, skin conditions, and nail brittleness. The methionine compounds are also useful for promoting or enhancing hair or fur growth in an animal. In particular, the methionine compound is generally a methionine derivative or methionine analog. The methionine compounds may be formulated with other agents known to be effective for any of the aforementioned conditions and may be administered to the subject by a variety of suitable means, including orally, topically, and by injection. An exemplary route for selective administration to hair follicle cells or skin cells is encapsulation of a composition of the invention within a suitable structured fluid delivery system.

### I. Methionine Analogs and Derivatives

One aspect of the invention encompasses compositions having at least one methionine derivative or methionine analog (hereinafter referred to as “methionine compound”). Methionine compounds suitable for use in compositions of the invention typically either promote hair growth or prevent hair loss. Alternatively, suitable methionine compounds may have efficacy against a variety of skin conditions and/or nail brittleness.

(a) Methionine Sulfoxides and Sulfones

In one embodiment, the methionine compound is a methionine sulfoxide or methionine sulfone having formula (I):

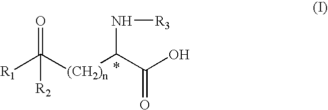

wherein:
* is a chiral carbon;
R₁ is methyl or ethyl;
R₂ is oxygen or hydrogen;
R₃ is an acyl group or hydrogen; and
n is an integer from 1 to 3.

Compounds corresponding to formula (I) may be a methionine sulfoxide (i.e., when R₂ is hydrogen) or a methionine sulfone (i.e., when R₂ is oxygen). Depending upon the embodiment, the compound having formula (I) may be normethionine (i.e., n is 1), methionine (i.e., n is 2) or homomethionine (i.e., n is 3). In certain embodiments, when R₃ is an acyl group, the compound having formula (I) may be an acetyl methionine sulfoxide or an acetyl methionine sulfone. Examples of suitable acyl groups include formyl, acetyl, propionyl and succinyl. Exemplary acyl groups are formyl and acetyl. The compound having formula (I) may also be an ester derivative. Examples of suitable ester derivatives include methyl, ethyl, propyl, isopropyl, and butyl esters. For each embodiment with compounds having formula (I), both the _{D}- and _{L}-isomers are included within the scope of the invention. The invention also encompasses pharmaceutically acceptable salts of compounds having formula (I). Suitable examples of salts include ammonium salt, alkaline earth metal salts (e.g., magnesium and calcium), alkali metal salts (e.g., lithium, sodium, and potassium), copper salts, zinc salts, cobalt salts, chromium salts, manganese salts, and iron salts.

In one embodiment, the compound having formula (I) is _{L}-methionine sulfoxide or _{D}-methionine sulfoxide (i.e., R₁ is methyl; R₂ is hydrogen, R₃ is hydrogen and n is 2). In an alternative embodiment, the compound having formula (I) is _{L}-methionine sulfone or _{D}-methionine sulfone (i.e., R₁ is methyl; R₂ is oxygen, R₃ is hydrogen and n is 2). In a further embodiment, the compound having formula (I) is N-acetyl-_{L}-methionine sulfoxide or N-acetyl-_{D}-methionine sulfoxide (i.e., R₁ is methyl; R₂ is hydrogen, R₃ is acetyl and n is 2). In another embodiment, the compound having formula (I) is N-formyl-_{L}-methionine sulfoxide or N-formyl-_{D}-methionine sulfoxide (i.e., R₁ is methyl; R₂ is hydrogen, R₃ is formyl and n is 2). In still another embodiment, the compound corresponding to formula (I) is N-acetyl-_{L}-methionine sulfone or N-acetyl-_{D}-methionine sulfone (i.e., R₁ is methyl; R₂ is oxygen, R₃ is acetyl and n is 2). In an additional embodiment, the compound corresponding to formula (I) is N-formyl-_{L}-methionine sulfone or N-formyl-_{D}-methionine sulfone (i.e., R₁ is methyl; R₂ is oxygen, R₃ is formyl and n is 2).

(b) Acyl Methionine Compounds

In an alternative embodiment, the methionine compound is an acyl methionine derivative having formula (II):

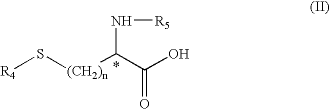

wherein:
* is a chiral carbon;
R₄ is methyl or ethyl;
R₅ is an acyl group;
n is an integer from 1 to 3.

The compound having formula (II) may be normethionine (i.e., n is 1), methionine (i.e., n is 2) or homomethionine (i.e., n is 3). Examples of suitable acyl groups (i.e., R₅) include formyl, acetyl, propionyl and succinyl. Exemplary acyl groups are formyl and acetyl. The compound having formula (II) may also be an ester derivative. Examples of suitable ester derivatives include methyl, ethyl, propyl, isopropyl, and butyl esters. For each embodiment with compounds having formula (II), both the _{D}- and _{L}-isomers are included within the scope of the invention. The invention also encompasses pharmaceutically acceptable salts of compounds having formula (II). Suitable examples of salts include ammonium salt, alkaline earth metal salts (e.g., magnesium and calcium), alkali metal salts (e.g., lithium, sodium, and potassium), copper salts, zinc salts, cobalt salts, chromium salts, manganese salts, and iron salts.

In one embodiment, the compound having formula (II) is N-acetyl-_{L}-methionine or N-acetyl-_{D}-methionine (i.e., R₄ is methyl; R₅ is acetyl and n is 2). In another embodiment, the compound having formula (I) is N-formyl-_{L}-methionine or N-formyl-_{D}-methionine (i.e., R₄ is methyl; R₅ is formyl and n is 2). In still another embodiment, the compound having formula (II) is N-propionyl-_{L}-methionine or N-propionyl-_{D}-methionine (i.e., R₄ is methyl; R₅ is propionyl and n is 2). In an additional exemplary embodiment, (i.e., R₁ is methyl; R₂ is oxygen, R₃ is formyl and n is 2). In a further embodiment, the compound having formula (II) is N-succinyl-_{L}-methionine or N-succinyl-_{D}-methionine (i.e., R₄ is methyl; R₅ is succinyl and n is 2).

(c) Peptides Having Methionine

In a further alternative embodiment, the methionine compound may include more than one methionine amino acid residue. In this context, the methionine compound may be a peptide that includes from about 1 to about 5 methionine amino acid residues. In an additional embodiment, the methionine compound may be a peptide that has from about 2 to about 4 methionine amino acid residues. In a further embodiment, the methionine compound will be a peptide having three methionine amino acid residues. In an exemplary embodiment, the methionine compound will be a dipeptide corresponding to formula (III):

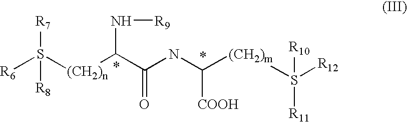

wherein:
* is a chiral carbon;
R₆ and R₁₂ are independently methyl or ethyl;
R₇, R₈, R₁₀ and R₁₁ are independently oxygen or hydrogen;
R₉ is an acyl group or hydrogen;
n is an integer from 1 to 3; and
m is an integer from 1 to 3.

Compounds corresponding to formula (III) may include one methionine sulfoxide group (e.g., one of R₇ or R₈ is hydrogen and one is oxygen) or two methionine sulfoxide groups (e.g., one of R₇ or R₈ is hydrogen and one is oxygen; and one of R₁₁ or R₁₂ is hydrogen and one is oxygen). Alternatively, compounds corresponding to formula (III) may include one methionine sulfone group (e.g., R₇ or R₈ are oxygen) or two methionine sulfone groups (e.g., R₇, R₈, R₁₀, and R₁₁ are oxygen). Depending upon the embodiment, the compound having formula (III) may include, one or two normethionines (i.e., n or m is 1), one or two methionines (i.e., n or m is 2) or one or two homomethionines (i.e., n or m is 3), and any combinations thereof. In certain embodiments, the compound having formula (III) may include an acyl group. Examples of suitable acyl groups include formyl, acetyl, propionyl and succinyl. Exemplary acyl groups are formyl and acetyl. The compound having formula (III) may also be an ester derivative. Examples of suitable ester derivatives include methyl, ethyl, propyl, isopropyl, and butyl esters. For each embodiment with compounds having formula (III), both the _{D}- and _{L}-isomers are included within the scope of the invention. In one embodiment the compound may be a _{D}-_{D}-isomer. In an alternative embodiment, the compound may be _{L}-_{L}-isomer. In a further embodiment, the compound may be a _{D}-_{L}-isomer. The invention also encompasses pharmaceutically acceptable salts of compounds having formula (III). Suitable examples of salts include ammonium salt, alkaline earth metal salts (e.g., magnesium and calcium), alkali metal salts (e.g., lithium, sodium, and potassium), copper salts, zinc salts, cobalt salts, chromium salts, manganese salts, and iron salts.

(d) Hydroxy Analogs of Methionine

In an exemplary embodiment, the methionine compound is a hydroxy analog of methionine. In one embodiment, the hydroxy analog of methionine is a compound having formula (IV):

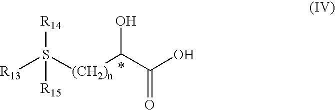

wherein:
is a chiral carbon;
R₁₃ is methyl or ethyl; and
R₁₄ and R₁₅ are independently oxygen or hydrogen.

Compounds corresponding to formula (IV) may be a methionine sulfoxide hydroxy analog (i.e., when one of R₁₄ or R₁₅ is hydrogen and one is oxygen) or a methionine sulfone hydroxy analog (i.e., when R₁₄ and R₁₅ are oxygen). The compound having formula (IV) may be normethionine (i.e., n is 1), methionine (i.e., n is 2) or homomethionine (i.e., n is 3). In an exemplary embodiment, the compound having formula (IV) is methionine. The compound having formula (IV) may also be an ester derivative. Examples of suitable ester derivatives include methyl, ethyl, propyl, isopropyl, and butyl esters. For each embodiment with compounds having formula (IV), both the _{D}- and _{L}-isomers are included within the scope of the invention. The invention also encompasses pharmaceutically acceptable salts of compounds having formula (IV). Suitable examples of salts include ammonium salt, alkaline earth metal salts (e.g., magnesium and calcium), alkali metal salts (e.g., lithium, sodium, and potassium), copper salts, zinc salts, cobalt salts, chromium salts, selenium salts, manganese salts, and iron salts.

In a further exemplary embodiment, the methionine compound is the hydroxyl analog of methionine corresponding to formula (V):

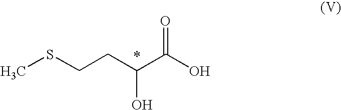

The compound having formula (V) is 2-hydroxy-4(methylthio)butanoic acid (commonly known as “HMTBA” and sold by Novus International, St. Louis, Mo. under the trade name Alimet®). A variety of HMTBA salts, chelates, esters, amides, and oligomers are also suitable for use in the invention. Representative salts of HMTBA, in addition to the ones described below, include the ammonium salt, the stoichiometric and hyperstoichiometric alkaline earth metal salts (e.g., magnesium and calcium), the stoichiometric and hyperstoichiometric alkali metal salts (e.g., lithium, sodium, and potassium), and the stoichiometric and hyperstoichiometric zinc salt. Representative esters of HMTBA include the methyl, ethyl, 2-propyl, butyl, and 3-methylbutyl esters of HMTBA. Representative amides of HMTBA include methylamide, dimethylamide, ethylmethylamide, butylamide, dibutylamide, and butylmethylamide. Representative oligomers of HMTBA include its dimers, trimers, tetramers and oligomers that include a greater number of repeating units.

Alternatively, the hydroxy analog of methionine may be a metal chelate comprising one or more ligand compounds having formula (IV) together with one or more metal ions. Irrespective of the embodiment, suitable non-limiting examples of metal ions include zinc ions, copper ions, manganese ions, iron ions, chromium ions, selenium ions, cobalt ions, and calcium ions. In one embodiment, the metal ion is divalent. Examples of divalent metal ions (i.e., ions having a net charge of 2⁺) include copper ions, manganese ions, calcium ions, cobalt ions and iron ions. In another embodiment, the metal ion is zinc. In yet another embodiment, the metal ion is copper. In still another embodiment, the metal ion is iron. In a further embodiment, the metal ion is calcium. In each embodiment, the ligand compound having formula (IV) is preferably HMTBA. In one exemplary embodiment, the metal chelate is HMTBA-Ca. In a further exemplary embodiment, the metal chelate is HMTBA-Cu. In an alternative exemplary embodiment, the metal chelate is HMTBA-Zn. In still another exemplary embodiment, the metal chelate is HMTBA-Fe.

As will be appreciated by a skilled artisan, the ratio of ligands to metal ions forming a metal chelate compound can and will vary. Generally speaking, where the number of ligands is equal to the charge of the metal ions, the charge of the molecule is typically net neutral because the carboxy moieties of the ligands having formula (IV) are in deprotonated form. By way of further example, in a chelate species where the metal ion carries a charge of 2⁺ and the ligand to metal ion ratio is 2:1, each of the hydroxyl group is believed to be bound by a coordinate covalent bond to the metal while an ionic bond exists between each of the carboxylate groups of the metal ion. This situation exists, for example, where divalent zinc, copper, or manganese is complexed with two HMTBA ligands. By way of further example, where the number of ligands exceeds the charge on the metal ion, such as in a 3:1 chelate of a divalent metal ion, the ligands in excess of the charge generally remain in a protonated state to balance the charge. Conversely, where the positive charge on the metal ion exceeds the number of ligands, the charge may be balanced by the presence of another anion, such as, for example, chloride, bromide, iodide, bicarbonate, hydrogen sulfate, and dihydrogen phosphate.

Generally speaking, a suitable ratio of ligand to metal ion is from about 1:1 to about 3:1 or higher. In another embodiment, the ratio of ligand to metal ion is from about 1.5:1 to about 2.5:1. Of course within a given mixture of metal chelate compounds, the mixture will include compounds having different ratios of ligand to metal ion. For example, a composition of metal chelate compounds may have species with ratios of ligand to metal ion that include 1:1, 1.5:1, 2:1, 2.5:1, and 3:1.

Metal chelate compounds of the invention may be made in accordance with methods generally known in the art, such as described in U.S. Pat. Nos. 4,335,257 and 4,579,962, which are both hereby incorporated by reference in their entirety. In a preferred process for the preparation of metal chelate compounds, a metal source compound, such as a metal oxide, a metal carbonate, or a metal hydroxide is charged to a reaction vessel, and an aqueous solution of HMTBA is added to the source compound. The concentration of HMTBA in the aqueous solution is typically about 40% to about 89% by weight. The reaction typically proceeds for a period of two hours under moderate agitation. Depending on the starting material used in the reaction, typically water and/or carbon dioxide are produced. Ordinarily, the reaction may be conducted at atmospheric pressure, and the reaction mass is heated to a temperature ranging from about 90° C. to about 130° C. After the reaction is substantially complete, heating of the reaction mass is continued in the reaction vessel to produce a substantially dried product. Typically, the free water content is reduced to about 2% by weight, and the product mass transitions to free-flowing particulate solid. The dried metal chelate product may optionally be mixed with a calcium bentonite filer, or silica as a and ground to a powder. Alternatively, the metal chelate compounds may be purchased from a commercially available source. For example, HMTBA-Zn and HMTBA-Cu may be purchased from Novus International, Saint Louis, Mo., sold under the trade names MINTREX® Zn, and MINTREX® Cu, respectively.

In an alternative exemplary embodiment, the hydroxy analog of methionine may be a pharmaceutically acceptable metal salt comprising an anionic compound having formula (IV) together with a metal ion. Typically, suitable metal ions will have either a 1⁺, 2⁺ or a 3⁺ charge and will be selected from zinc ions, copper ions, manganese ions, selenium ions, iron ions, chromium ions, silver ions, cobalt ions, and silver ions. Without being bound by any particular theory, however, it is generally believed that combinations of zinc, copper, manganese, iron, selenium, chromium, nickel, and cobalt ions together with HMTBA form metal chelates as opposed to salts. Irrespective of whether the molecule formed is a salt or a chelate, both forms of the molecules are included within the scope of the invention. Salts useful in the invention may be formed when the metal, metal oxide, metal hydroxide or metal salt (e.g., metal carbonate, metal nitrate, or metal halide) react with one or more compounds having formula (IV). In an exemplary embodiment, the compound having formula (IV) will be HMTBA.

Salts may be prepared according to methods generally known in the art. For example, a metal salt may be formed by contacting HMTBA with a metal ion source. In one embodiment, a silver ion having a 1+charge may be contacted with HMTBA to form a silver 2-hydroxy-4-methylthiobutanoate metal salt. This salt generally will have a silver to HMTBA ratio of approximately 1:1.

(e) Blends of Methionine Compounds

The compositions of the invention include at least one methionine compound. It is also envisioned that certain compositions may include combinations of two or more of any of the methionine compounds detailed herein or otherwise known in the art. In some embodiments, the composition may include two methionine compounds. In other embodiments, the composition may include three methionine compounds. In additional embodiments, the composition may include four or more methionine compounds. Non-limiting examples of suitable compositions having more than one methionine compound are set-forth in Table A.

TABLE A

| COMPOUND NO. 1 | COMPOUND NO. 2 |
|---|---|
| A compound having formula (I) | A compound having formula (II) |
| A compound having formula (I) | A compound having formula (III) |
| A compound having formula (I) | A compound having formula (IV) |
| A compound having formula (I) | HMTBA |
| A compound having formula (I) | HMTBA-ester |
| A compound having formula (I) | HMTBA-Cu |
| A compound having formula (I) | HMTBA-Zn |
| A compound having formula (I) | HMTBA-Ca |
| A compound having formula (I) | HMTBA-Fe |
| A compound having formula (I) | HMTBA-Se |
| A compound having formula (I) | HMTBA-Mn |
| A compound having formula (I) | HMTBA-Cr |
| A compound having formula (I) | HMTBA-Ag |
| A compound having formula (II) | A compound having formula (III) |
| A compound having formula (II) | A compound having formula (IV) |
| A compound having formula (II) | HMTBA |
| A compound having formula (II) | HMTBA-ester |
| A compound having formula (II) | HMTBA-Cu |
| A compound having formula (II) | HMTBA-Zn |
| A compound having formula (II) | HMTBA-Ca |
| A compound having formula (II) | HMTBA-Fe |
| A compound having formula (II) | HMTBA-Se |
| A compound having formula (II) | HMTBA-Mn |
| A compound having formula (II) | HMTBA-Cr |
| A compound having formula (II) | HMTBA-Ag |
| A compound having formula (III) | A compound having formula (II) |
| A compound having formula (III) | HMTBA |
| A compound having formula (III) | HMTBA-ester |
| A compound having formula (III) | HMTBA-Cu |
| A compound having formula (III) | HMTBA-Zn |
| A compound having formula (III) | HMTBA-Ca |
| A compound having formula (III) | HMTBA-Fe |
| A compound having formula (III) | HMTBA-Se |
| A compound having formula (III) | HMTBA-Mn |
| A compound having formula (III) | HMTBA-Cr |
| A compound having formula (III) | HMTBA-Ag |
| HMTBA | HMTBA-ester |
| HMTBA | HMTBA-Cu |
| HMTBA | HMTBA-Zn |
| HMTBA | HMTBA-Ca |
| HMTBA | HMTBA-Fe |
| HMTBA | HMTBA-Se |
| HMTBA | HMTBA-Mn |
| HMTBA | HMTBA-Cr |
| HMTBA | HMTBA-Ag |
| HMTBA-ester | HMTBA-Cu |
| HMTBA-ester | HMTBA-Zn |
| HMTBA-ester | HMTBA-Ca |
| HMTBA-ester | HMTBA-Fe |
| HMTBA-Cu | HMTBA-Zn |
| HMTBA-Cu | HMTBA-Ca |
| HMTBA-Cu | HMTBA-Fe |
| HMTBA-Zn | HMTBA-Ca |
| HMTBA-Zn | HMTBA-Fe |
| HMTBA-Ca | HMTBA-Fe |
| HMTBA and/or HMTBA-ester | HMTBA-Cu, HMTBA-Zn, |
| | HMTBA-Ca, and HMTBA-Fe |
| HMTBA and/or HMTBA-ester | HMTBA-Cu, HMTBA-Zn, and |
| | HMTBA-Ca, |
| HMTBA and/or HMTBA-ester | HMTBA-Cu and HMTBA-Zn |
| HMTBA and/or HMTBA-ester | HMTBA-Cu and HMTBA-Ca |
| HMTBA and/or HMTBA-ester | HMTBA-Cu and HMTBA-Fe |
| HMTBA and/or HMTBA-ester | HMTBA-Zn, HMTBA-Ca, and |
| | HMTBA-Fe |
| HMTBA and/or HMTBA-ester | HMTBA-Zn and HMTBA-Ca |
| HMTBA and/or HMTBA-ester | HMTBA-Zn and HMTBA-Fe |
| HMTBA and/or HMTBA-ester | HMTBA-Ca and HMTBA-Fe |

The compositions of the invention, including any detailed herein such as in Table A, have utility as hair growth agents. The compositions of the invention may also be used, in certain embodiments, to control dandruff. More particularly, the compositions may be utilized to stimulate hair growth on warm-blooded subjects. As such, the compositions may be used to treat a variety of diseases states or disorders associated with hair loss arising from any of a variety of forms of primary alopecia, including (but not limited to) androgenetic alopecia (also know as male pattern baldness), alopecia areata and female pattern baldness. In these instances, the compositions of the invention typically stimulate the growth of hair after the onset of the hair-loss affliction. Alternatively, the compositions of the invention may be administered prophylactically for conditions such as secondary alopecia. For example, the compositions may be administered prior to an insult that normally results in hair loss, such as chemotherapy and/or radiation treatment. The composition may also be utilized, as demonstrated in example 2, to promote coat quality or growth in animals.

In an additional aspect of the invention, the compositions have utility for treating nail brittleness and/or a variety of skin conditions. For example, the compositions of the invention may be utilized in methods for treating mammalian skin to condition and smoothen the skin, lessen hyperpigmentation, and prevent or reduce the appearance of fine lines and wrinkles, and other signs of photodamage and aging of the skin. The compositions may also be utilized to treat a variety of skin disorders, such as excessive dryness or acne. The method comprises contacting the skin with an effective amount of an above-disclosed composition. As a specific example, a small amount of material (from about 0.1 to about 5 ml) is applied to exposed areas of skin from a suitable container or applicator, and, if necessary, the material is then spread over and/or rubbed into the skin using the hand or finger, or a suitable device. Each of the compositions and preparations disclosed herein is typically packaged in a container to suit its viscosity and intended use by the subject. For example, a lotion or fluid cream may be packaged in a bottle, roll-ball applicator, capsule, propellant-driven aerosol device, or a container fitted with a manually operated pump. A cream can simply be stored in a non-deformable bottle or squeeze container, such as a tube or a lidded jar. A variety of suitable formulations of compositions of the invention for hair, skin, and nail treatment are detailed in section II below more thoroughly.

Generally speaking, the compositions of the invention are administered to a subject in need thereof in a manner that results in the application of an effective amount of the composition. As will be appreciated by a skilled artisan, the amount comprising an “effective amount” can and will vary depending upon the indication being treated, the age and sex of the subject, the formulation of the composition, and the presence of other active agents combined with the composition of the invention. Typically, any of the methionine compounds detailed above, either alone or in combination, may be present in the composition in an amount ranging from about 0.01% to about 25% by weight. In another embodiment, the methionine compound(s) may be present in the composition in an amount ranging from about 0.01% to about 10% by weight. In a further embodiment, the methionine compound(s) may be present in the composition in an amount ranging from about 0.01% to about 5% by weight. Suitable formulations and routes of administration are described in section II and III below. Suitable combination treatments are detailed in section IV below.

### II. Formulations and Routes of Administration

The composition comprising methionine compounds may be administered by a variety of methods known in the art that will result in the delivery, including delivery to hair follicles, nails or skin, of an effective amount of the composition. As will be appreciated by a skilled artisan, the method of such administration can and will vary depending upon the particular composition, the indication, and the condition of the subject. Such compositions, for example, can be administered orally, parenterally, by spray, rectally, intradermally, transdermally, or topically in dosage unit formulations containing conventional nontoxic pharmaceutically acceptable carriers, adjuvants, and vehicles as desired. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

(a) Oral Administration

The methionine compounds may be formulated for oral administration. Solid dosage forms for oral administration may include capsules, tablets, pills, powders, and granules. In such solid dosage forms, the compound is ordinarily combined with one or more adjuvants appropriate to the indicated route of administration. The compound can be admixed with various carbohydrate fillers/binders, coating materials, lubricants, disintegrants, flavors, coloring agents such as lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

Alternatively, the composition may be administered as a liquid. Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of the methionine compound that can be combined with the carrier materials to produce a single oral dosage of the composition will vary depending upon the patient and the particular mode of administration. Generally speaking, the dosage may contain from 0.005% to about 20% by weight of any of the methionine compounds detailed herein along with from about 80% to about 99% by weight of any of the ingredients acceptable for oral formulations as detailed herein or otherwise known in the art. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

(b) Topical Administration

In another aspect of the invention, the methionine compounds are formulated for topical administration. Topical compositions that can be applied locally to the skin may be in any form including solutions, oils, creams, liquids, ointments, gels, lotions, shampoos, leave-on and rinse-out hair conditioners, milks, cleansers, moisturizers, sprays, skin patches, and the like. Topical administration may be accomplished by application directly on the treatment area. For example, such application may be accomplished by rubbing the formulation (such as a lotion or gel) onto the skin of the treatment area, or by spray application of a liquid formulation onto the treatment area. In an exemplary embodiment, as detailed more thoroughly in section III. below, topical delivery is achieved by incorporating the methionine compounds into a structured fluid delivery vesicle.

Topical administration may also involve the use of transdermal administration such as transdermal patches or iontophoresis devices. For example, the methionine compounds may be formulated in an instrument adapted to deliver them to hair follicles via iontophoresis. As is appreciated by one skilled in the art, such a formulation is typically in the form of a liquid (i.e., solution), rather than a cream or gel. An example of an instrument adapted for such delivery is a large bandage comprising a chamber and delivering an electrical current. The chamber is situated so as to be in contact with the skin and comprises the formulation. In a further embodiment, the methionine compounds are formulated for delivery to hair follicles via ultrasound. It is generally believed, without being bound by any particular theory, that ultrasound and iontophoresis enhance the delivery of the methionine compounds to the hair follicles by disturbing the stratum corneum, thereby improving the transport of the active compounds.

As will be appreciated by a skilled artisan, the topical formulation in addition to the methionine compounds, may include a variety of suitable inert, physiologically acceptable carriers and diluents. Suitable carriers or diluents include, but are not limited to, water, physiological saline, bacteriostatic saline (saline containing 0.9 mg/ml benzyl alcohol), petrolatum based creams (e.g., USP hydrophilic ointments and similar creams, Unibase, Parke-Davis, for example), various types of pharmaceutically acceptable gels, and short chain alcohols and glycols (e.g., ethyl alcohol and propylene glycol).

In addition to carriers and diluents, topical formulations may optionally contain additional agents such as penetration enhancement agents, surface-active agents, emollients, propellants, solvents, humectants, thickeners, powders, and fragrances (i.e., “additional agents”). As will be appreciated by a skilled artisan, the topical formulation may include a combination of any of the forgoing additional agents in varying amounts. Generally, the topical formulation will comprise one or more methionine compounds in an amount ranging from 0.1% to 20% by weight of the composition and from about 80% to about 99% by weight of any combination of additional agents. In a further embodiment, the topical formulation will comprise one or more methionine compounds in an amount ranging from 0.1% to 5% by weight of the composition and from about 95% to about 99% by weight of any combination of additional agents. Suitable examples of each type of additional agent are detailed below.

The topical formulation may optionally include one or more surface-active agents (also called emulsifying agents). Emulsifiers and surfactants are generally used in preparing those embodiments of the present invention directed to compositions that are formulated as emulsions. Either water in oil or oil in water emulsions may be formulated. Examples of suitable surfactants and emulsifying agents include: nonionic ethoxylated and nonethoxylated surfactants, abietic acid, almond oil PEG, beeswax, butylglucoside caprate, C₁₈-C₃₆ acid glycol ester, C₉-C₁₅ alkyl phosphate, caprylic/capric triglyceride PEG-4 esters, ceteareth-7, cetyl alcohol, cetyl phosphate, corn oil PEG esters, DEA-cetyl phosphate, dextrin laurate, dilaureth-7 citrate, dimyristyl phosphate, glycereth-17 cocoate, glyceryl erucate, glyceryl laurate, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPG's, raffinose myristate, sodium caproyl lactylate, sodium caprylate, sodium cocoate, sodium isostearate, sodium tocopheryl phosphate, steareths, TEA-C₁₂-C₁₃ pareth-3 sulfate, tri-C₁₂-C₁₅ pareth-6 phosphate, and trideceths.

In an additional embodiment, the topical formulation may include one or more penetration enhancing agents to increase absorption across the skin. Exemplary penetration enhancing agents include dimethyl sulfoxide (DMSO), urea and substituted urea compounds. Examples of other suitable penetration enhancers include 2-methyl propan-2-ol, propan-2-ol, ethyl-2-hydroxypropanoate, hexan-2,5-diol, polyoxyethylene(2) ethyl ether, di(2-hydroxypropyl)ether, pentan-2,4-diol, acetone, polyoxyethylene(2) methyl ether, 2-hydroxypropionic acid, 2-hydroxyoctanoic acid, propan-1-ol, 1,4-dioxane, tetrahydrofuran, butan-1,4-diol, propylene glycol dipelargonate, polyoxypropylene 15 stearyl ether, octyl alcohol, polyoxyethylene ester of oleyl alcohol, oleyl alcohol, lauryl alcohol, dioctyl adipate, dicapryl adipate, di-isopropyl adipate, di-isopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hydroxyoctanoic acid, dimethyl sulphoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, 1-dodecylazacyloheptan-2-one, omega three fatty acids and fish oils, and combinations thereof.

In an additional embodiment, the topical formulation may optionally include one or more emollients. An emollient, as the term is used herein, is a cosmetic ingredient that can help skin maintain a soft, smooth, and pliable appearance. Examples of suitable emollients include, acetyl arginine, acetylated lanolin, algae extract, apricot kernel oil PEG-6 esters, avocado oil PEG-11 esters, bis-PEG-4 dimethicone, butoxyethyl stearate, C₁₈-C₃₆ acid glycol ester, C₁₂-C₁₃ alkyl lactate, caprylyl glycol, cetyl esters, cetyl laurate, coconut oil PEG-10 esters, di-C₁₂-C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, hydroxypropyl bisisostearamide MEA, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, odontella aurita oil, 2-oleamido-1,3 octadecanediol, palm glycerides, PEG avocado glycerides, PEG castor oil, PEG-22/dodecyl glycol copolymer, PEG shorea butter glycerides, phytol, raffinose, stearyl citrate, sunflower seed oil glycerides, and tocopheryl glucoside.

The topical formulation also may optionally include a humectant. Humectants are cosmetic ingredients that generally help maintain moisture levels in skin. Suitable examples of humectants include, acetyl arginine, algae extract, aloe barbadensis leaf extract, betaine, 2,3-butanediol, chitosan lauroyl glycinate, diglycereth-7 malate, diglycerin, diglycol guanidine succinate, erythritol, fructose, glucose, glycerin, honey, hydrolyzed wheat protein/PEG-20 acetate copolymer, hydroxypropyltrimonium hyaluronate, inositol, lactitol, maltitol, maltose, mannitol, mannose, methoxy PEG, myristamidobutyl guanidine acetate, polyglyceryl sorbitol, potassium PCA, propylene glycol, sodium PCA, sorbitol, sucrose, and urea.

In certain applications, it may be desirable to thicken the topical formulation. Suitable examples of thickening or viscosity increasing agents, include agents such as: acrylamides copolymer, agarose, amylopectin, bentonite, calcium alginate, calcium carboxymethyl cellulose, carbomer, carboxymethyl chitin, cellulose gum, dextrin, gelatin, hydrogenated tallow, hydroxytheylcellulose, hydroxypropylcellulose, hydroxpropyl starch, magnesium alginate, methylcellulose, microcrystalline cellulose, pectin, various PEG's, polyacrylic acid, polymethacrylic acid, polyvinyl alcohol, various PPG's, sodium acrylates copolymer, sodium carrageenan, xanthan gum, and yeast beta-glucan.

For topical applications that are formulated as a spray, the composition will generally include a propellant. Suitable propellants include propane, butane, isobutane, dimethyl ether, carbon dioxide, nitrous oxide, and combinations thereof.

In an additional embodiment, the topical agent may include one or more solvents. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. An exemplary solvent is ethyl alcohol.

In a further embodiment, the topical formulation may optionally include one or more powders. Suitable powders include chalk, talc, fullers earth, kaolin, starch, gums, colloidal silicon dioxide, sodium polyacrylate, tetra alkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

(c) Administration by Injection

In another aspect of the invention, the methionine compounds may be administered by injection, such as intradermal injection, into the treatment area, including directly into hair follicles. While the injection is typically intradermal, other suitable modes of injection include subcutaneous, intravenous, intramuscular, or by infusion techniques.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The methionine compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

### III. Structured Fluid Delivery Systems

In an exemplary embodiment, the present invention also encompasses use of a structured fluid system to selectively deliver methionine compounds to hair follicles or skin cells. In this context the word “selectively” means that the methionine compounds are substantially delivered directly to the hair follicle or skin cells as opposed to delivery either to surrounding cells that are not hair follicle or skin cells or delivery to the circulatory system. In this embodiment, typically a composition of the invention is encapsulated in a suitable structured fluid delivery system to either aid in the delivery of the compound to hair follicles or skin cells or to increase the stability of the composition. As will be appreciated by a skilled artisan, a variety of structured fluid delivery systems are suitable for use in the present invention. Examples of suitable structured fluid delivery systems include liposomes, microemulsions, micelles, dendrimers and other phospholipid-containing systems.

(a) Liposome Delivery System

In one alternative embodiment, a liposome delivery system may be utilized. Liposomes, depending upon the embodiment, are suitable for delivery of the composition of the invention in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the composition of the invention may be selectively delivered to a hair follicle or skin cell by encapsulation in a liposome that fuses with the targeted cell's membrane.

It is envisioned that the liposome may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unstaurated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palm itoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). The pegylated lipid PEGs may generally increase the amount of compound that can be incorporated into the liposomes. PEGs may range in size from about 500 to about 10,000 daltons. A suitable pegylated phospholipid is dipalmitoyl PE bearing PEG 5,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the composition of the invention (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. In one embodiment, for example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes. In a preferred embodiment, at least a majority of the lipsomes are small unilamellar liposomes. In an exemplary embodiment, at least 90% of the liposomes are small unilamellar liposomes. Small unilamellar liposomes may be enriched for by filtering the liposomal suspension and/or mechanically extruding the suspension through a small aperature, whereby the majority of liposomes in the final preparation are small and unilamellar.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

(b) Microemulsion Delivery System

In another embodiment, the composition of the invention may be delivered to hair follicle or skin cells as a microemulsion. Microemulsions are generally clear, thermodynamically stable solutions comprising an aqueous solution, a surfactant, and “oil.” The “oil” in this case, is the supercritical fluid phase. A suitable oil for use in the invention is a supercritical fluid that has the unique ability to diffuse through solids like a gas, and dissolve material like a liquid. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The composition of the invention may be encapsulated in a microemulsion by any method generally known in the art.

(c) Dendrimer Delivery System

In yet another embodiment, the composition of the invention may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of an dendrimer to facilitate absorption across the skin. In an exemplary embodiment, the dedrimer will have a phospholipid disposed on its surface to aid transport across the skin or hair follicle cell. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### IV. Combination Therapy

It is also envisioned that the methionine compounds of the invention may be combined with a variety of other active agents known in the art to be effective for treating alopecia, skin, or nail brittleness or for promoting hair growth. For example, the additional agent, when formulated with the composition of the invention, may act synergistically to enhance the treatment of alopecia, skin, or nail brittleness. A variety of suitable additional active agents are detailed below.

One aspect of the invention encompasses use of an antioxidant in combination with a methionine compound. In one alternative, the antioxidant agent may be one or more vitamins that have antioxidant activity, or a combination of a vitamin having antioxidant activity along with one of several other vitamins that are administered to the subject to meet nutritional requirements. In one alternative of this embodiment, the vitamin is tocopherol, commonly known as vitamin E. Suitable forms of tocopherals include, alpha-, beta-, gamma- or delta-tocopherol, and its esters, especially vitamin E (tocopherol acetate), tocopheryl succinate, tocopherylnicotinate or tocopherylpoly(oxyethylene)-succinate. Another suitable tocopheral is the desmethyl tocopherols detailed in U.S. Pat. No. 6,346,544, which is hereby incorporated by reference in its entirety. In another alternative of this embodiment, the vitamin is ascorbic acid, commonly known as vitamin C. In yet another alternative embodiment, the antioxidant agent is a carotenoid, such as from vitamin A. A number of different carotenoids may be employed in the composition. By way of example, the carotenoid may be beta-carotene. By way of further example, the carotenoid may be lycopene. Other vitamins suitable for use in the invention include, without limitation, nicotinic acid esters, benzyl nicotinate and C₁-C₆ alkyl nicotinates, such as methyl nicotinate or hexyl nicotinate; nicotinic acid esters, including in particular tocopherol nicotinate, benzyl nicotinate and C₁-C₆ alkyl nicotinates, such as methyl nicotinate or hexyl nicotinate; imidine derivatives, such as 2,4-diamino-6-piperidinopyrimidine 3-oxide, B₁ (thiamin), B₂ (riboflavin), B₃ (niacin), B₅ (pantothenate), B₇ (biotin, vitamin H), B₉ (folate), B₁₀, B₁₂, vitamin K (menadione), pyrimidine N oxides, PABA (para-amino butyric acid), choline, and inositol.

In a further alternative, the methionine compound(s) is combined with one or more enzymes or coenzymes that are effective in treating alopecia, skin, or nail brittleness. In certain embodiments, the enzyme or coenzyme may be combined with one or more of the vitamins detailed herein or otherwise known in the art. A number of coenzymes may be utilized in the composition. One example of a suitable coenzyme is coenzyme Q, also known as ubiquinone. In an alternative embodiment, the coenzyme may be an analogue of coenzyme Q. A suitable analogue of coenzyme Q is idebenone. By way of further example, the coenzyme may be lipoic acid. Other suitable enzymes or coenzymes include, superoxide dismutases (metal binding peptides), cocarboxylases, coenzyme A (aka pantothenate or B5), NAD, FAD, NADP, glutathione, and bioflavonoids.

In an additional embodiment, the agent may be an antioxidant isolated from a natural product or is a herb. Natural products suitable for use in the composition of the invention include food sources or compositions isolated from food sources that have antioxidant activity. In one embodiment, the natural product is an extract made from the dried leaves of a ginkgo biloba tree. A number of different variants of ginkgo biloba extracts are commercially available including Ginkgold (EGb 761), LL 1369, and Chinese Ginkgo extract ZGE. Alternatively, the ginkgo biloba may be extracted from dried leaves by any generally known method as detailed in U.S. Pat. No. 6,447,819, which is hereby incorporated by reference in its entirety. In a yet another embodiment, the natural product is a phytoalexin isolated from a plant. In one embodiment, the phytoalexin is resveratrol or an isoform or derivative thereof. While present in a number of plants, such as eucalyptus, spruce, and lily, and in other foods such as mulberries and peanuts, resveratrol's most abundant natural sources are vitis vinifera, labrusca, and muscadine grapes, which are used to make red wines. Other suitable herbs include, without limitation, gotu kola, muira puma, cucurbita maxima (pumpkin seeds), saw palmetto, radix urticae (stinging nettle root), eleuthero, and Uva-Ursi.

In a further embodiment, the methionine compound may be administered with amino acids, amino acid analogs or derivatives of amino acids. An amino acid of this invention includes any carboxylic acid having an amino moiety, including (but not limited to) the naturally occurring alpha-amino acids (in the following listing, the single letter amino acid designations are given in parentheses): alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), serine (S), threonine (T), tryptophan (W), tyrosine (Y) and valine (V). Other naturally occurring amino acids include (but are not limited to) hydroxyproline and gamma-carboxyglutamate. In a preferred embodiment, the amino acid is a naturally occurring alpha-amino acid having an amino moiety (i.e., the —NH₂ group, rather than a secondary amine, —NH—, such as present in proline). In one embodiment, the amino acid is selected from methionine, lysine, arginine, cysteine, taurine, tyrosine. In an exemplary embodiment, the amino acid is a cysteine, cysteine derivative or cysteine analog. While the chiral amino acids of the present invention have not been specifically designated, the present invention encompasses both the naturally occurring _{L}-form, as well as the _{D}-form.

In an additional embodiment, the methionine compounds are combined with one or more commercially available topical drugs known to be effective for treating alopecia. In one embodiment, the topical drug is minoxidil (i.e., sold under the trade name Rogaine® by UpJohn Company previously, and a Pfizer Co recently). In an alternative embodiment, the topical drug is finasteride (i.e., sold under the trade name Propecia® by Merck and Company). In an additional embodiment, the topical drug is selected from the group consistent of revivogen, avodart, fluridil, proscar, retin A, and fabio 101.

A further aspect of the invention encompasses combining a methionine compound with one or more metal ions effective for treating alopecia, skin, or nail brittleness. In one alternative of this embodiment, the metal ion is zinc. In an additional alternative of this embodiment, the metal ion is selenium. In still another alternative of this embodiment, the metal ion is copper. In a further alternative of this embodiment, the metal ion is magnesium. In an additional alternative embodiment, the metal ion is calcium. In another embodiment, the metal ion may form a complex of metal ions. In one alternative of this embodiment, the complex is a platinum-based complex. In an additional alternative of this embodiment, the complex is a peptide-copper complex.

A variety of other agents known to be effective for treating alopecia, skin, or nail brittleness may also be combined with the methionine compound(s). For example, the active agent may be selected from among antibacterial agents (e.g., macrolides, pyranosides and tetracyclines, and erythromycin), agents for combating parasites (e.g., metronidazole, crotamiton or pyrethroids), antifungal agents (e.g., compounds belonging to the imidazole class, such as econazole, ketoconazole or miconazole or their salts, polyene compounds, such as amphotericin B, compounds of the allylamine family, such as terbinafine, or alternatively octopirox), antiviral agents (e.g., acyclovir), anti-inflammatory agents (e.g., hydrocortisone, betamethasone valerate or clobetasol propionate, or nonsteroidal anti-inflammatory agents, such as ibuprofen and its salts, diclofenac and its salts, acetylsalicylic acid, acetaminophen or glycyrrhetinic acid), antipruriginous agents (e.g., thenaldine, trimeprazine or cyproheptadine), anaesthetic agents (e.g., lidocaine hydrochloride and derivatives thereof), keratolytic agents (e.g., alpha- and beta-hydroxycarboxylic acids or beta-ketocarboxylic acids, their salts, amides or esters and more particularly hydroxy acids, such as glycolic acid, lactic acid, salicylic acid, citric acid and, generally, fruit acids, and 5-(n-octanoyl)salicylic acid), agents for combating free radicals (e.g., superoxide dismutase or dimethyl sulfoxide), antiseborrhoeic agents (e.g., progesterone), antidandruff agents (e.g., octopirox or zinc pyrithione), antiacne agents (e.g., retinoic acid or benzoyl peroxide), agents which modulate cutaneous pigmentation and/or proliferation and/or differentiation, bradykinin antagonists, lamin, polysorbate 80, dimethylglycine (DMG), methylsulfonylmethane (MSM), antioxidants (e.g., BHT), vasodilating substance, chelating agents (e.g., EDTA), buffers (e.g., phosphate and tris(hydroxyaminomethane)), calcium antagonists (e.g., cinnarizine and diltiazem), hormones, (e.g., estriol or analogs thereof, or thyroxine and its salts), steroidal anti-inflammatory agents, (e.g., corticosteroids), antiandrogen agents (e.g., oxendolone, spironolactone or diethylstilbestrol), and 5-alpha-reductase antagonists.

### Definitions

The term “alopecia” as used herein refers to the loss of hair, wool, or feathers by a mammal-irrespective of the reason for the loss.

The terms “hair,” “fur,” and coat are used interchangeably herein.

The term “nail brittleness” is used in its broadest meaning herein to refer to the inability to grow long nails or the characterization of nails as dry, weak, easily splitable, or easily breakable. More objective clinical features seen in brittle nails are onychoschizia (transverse splitting), onychorrhexis (longitudinal splitting), and nail plate surface degranulation. Brittle nails may also be found in certain skin diseases such as psoriasis, lichen planus, and alopecia arcata.

The term “pharmaceutically-acceptable salts,” as used herein, are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine-(N-methylglucamine) and procaine. All of these salts may be prepared by conventional means from the corresponding compound by reacting, for example, the appropriate acid or base with any of the methionine compounds set forth herein.

“Skin condition” is used herein in its broadest sense to encompass any of a variety of skin diseases, disorders, discomforts or abnormalities for which any of the compositions of the invention are effective for treating. These conditions may include, for example, any altered state of the epidermis (e.g., rashes, blisters, itching, discoloration) caused by bacterial, fungal or viral infections, sunlight or other environmental factors, genetic predispositions, or factors of unknown origin. Examples of skin disorders include various forms of acne, dermatitis, eczema, pityriasis, and psoriasis, as well as couperose, ichthyosis (excessively dry skin), rosacea, solar keratosis, sunburn, and xanthelasma

“Subject” as used herein means a mammal in need of treatment, such as a mammal that has been diagnosed by any method generally known in the art as having alopecia, nail brittleness or a skin condition. In an exemplary embodiment, the subject is a human in need of treatment for alopecia, skin, or nail brittleness. In another exemplary embodiment, the subject has been diagnosed with alopecia, a skin condition or nail brittleness. In addition, the subject may be a mammal where it is desired to enhance or promote hair growth. Non-limiting examples of mammals include humans, primates, goats, sheep, pigs, cows, canids (e.g., dogs, foxes, coyotes, and wolves), rodents (e.g., mink, chinchillas, rabbits, raccoons, and beavers), and equids (e.g., horses, zebras, donkeys, and mules).

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate the invention. In particular, example 1 demonstrates methods for testing use of compositions to promote hair growth in a murine model for alopecia. Example 2 demonstrates methods for testing compositions to promote or enhance coat growth in dogs.

### Example 1

### Topical Application of Liposome Compositions to Stimulate Hair Growth in SKH-1 Hairless Mice

SKH-1 mice, an uncharacterized/non-pedigreed hairless strain of mice, are routinely used in various types of dermal research. These mice, therefore, provide an excellent system in which stimulation of hair growth may be examined. Accordingly, the liposome compositions of the invention comprising a metal chelate of a hydroxy analog of methionine may be tested in SKH-1 mice for their ability to treat alopecia by stimulating hair growth.

Mice. Two groups of 5-wk-old SKH-1 hairless mice (Charles River, Boston, Mass.) matched for age, size, and sex may be utilized in this study. Each mouse in the test group may be treated with a daily application of about 20-30 μL of a liposome composition applied topically with a plastic spatula to a defined 3×3 cm region in the midline of the back for 7 days. The control group may be treated with vehicle alone.

Measurement of mouse hair characteristics. After 1 week, the animals may be sacrificed and full-thickness dorsal skin samples taken. Samples may be fixed in formalin, embedded in paraffin, and longitudinally sectioned. Random 2-mm segments of epidermis may be delineated on the histology sections (stained with hematoxylin and eosin), such that dermal and epidermal thickness may be measured, and the number of hairs protruding may be counted. To assess hair length, the mice may be photographed prior to sacrifice, and 10 random hairs visible in cross-section may be measured.

Evaluation of mouse epidermal proliferation. Epidermal proliferation may be assessed by measuring ³H-thymidine incorporation and 5-bromo-2′-deoxyuridine (BrdU; Roche Molecular Biochemicals, Mannheim, Germany) incorporation into newly synthesized DNA. Twenty-four hours prior to sacrifice, each mouse may be injected intraperitonealy with ³H-thymidine, and three hours prior to sacrifice, each animal may be injected with additional ³H-thymidine along with BrdU. ³H-thymidine incorporation into DNA may be assessed with a β-counter and corrected for DNA content. Skin samples described above may be stained for BrdU (Isseroff et al., 1989, Br J Dermatol 120:503-510. BrdU data may be reported as the number of cell nuclei stained per millimeter epidermis or the percentage of hair follicles staining positive for BrdU.

Statistical analysis. A statistical analysis of the data may be performed with Student's t test, and the data may be presented ±standard error of the mean (SEM).

### Example 2

### Oral Ingestion of Compositions to Improve Hair Coat in Dogs

The objective of this study is to assess the effect of the supplementation of chelated organic or inorganic minerals in dog diets on immune parameters and on the mineral content of the hair coat of dogs of different breeds. Accordingly, a diet supplemented with an organic trace mineral blend (comprising Zn, Mn, Se, and Cu as a methionine hydroxy analog blend) may be compared with a diet supplemented with the inorganic trace minerals (added at the same level) with respect to the effects on humoral immunity in adult dogs, Zn deposition in the hair coat, and serum concentrations of these trace minerals.

Diets. Two individual diets may be manufactured that differ only in the form of the added trace minerals. The levels of Zn, Cu, and Mn are the same as those commonly used in commercial diets. “Control” Diet A—supplemented with inorganic trace minerals and DL-Met to the methionine level corresponding to that supplied by the inclusion of the hydroxy analog of methionine (HMTBA) in Diet B. “Experimental” Diet B—supplemented with organic trace minerals, Zn, Mn, Se, and HMTBA-Cu, at the same level as Diet A. The other minerals added in the inorganic form.

The basal diet may have the following formulation (Table 1).

TABLE 1
Basal Diet

| Ingredients | kg/MT |
|---|---|
| Ground corn (grain) | 150.00 |
| Linseed - grain | 4.00 |
| Broken rice | 325.00 |
| Wheat bran | 100.00 |
| Brewer's yeast | 1.00 |
| Poultry fat | 30.00 |
| Soybean oil | 15.00 |
| Meat meal 45% | 120.00 |
| Offal meal 50% | 210.00 |
| Salt - sodium chloride | 5.00 |
| Poultry meal | 19.60 |
| NOVUS Min. Vit Premix. | 10.00 |
| Yucca | 0.40 |
| Liquid Caramel Color | 10.00 |
| Total Weight | 1000.00 |

Calculated Composition

| | |
|---|---|
| Crude Protein % | 24.00 |
| ME Kcal/Kg | 3.500 |
| Ether extract % | 11.00 |
| Ca % | 2.00 |
| P % | 1.20 |
| Crude fiber % | 2.20 |
| Ashes % | 7.40 |
| Moisture % | 12.00 |

Two mineral premixes may be manufactured: a) 100% inorganic trace minerals+DL-Met; and b) organic Zn, Mn, Se, and HMTBA-Cu, with the remaining trace minerals added as inorganic. A single batch of feedstuffs may be prepared and divided (A and B), and the premixes will be added and mixed. Before the beginning of the trial, diets may be analyzed to check for the nutritional levels of Zn, Cu and Mn, and the presence of HMTBA.

Animals and experimental design. Twenty dogs from different breeds (German Shepherd, Malinois Shepherd, Labrador, or Rottweiler), 2 to 4 years of age, may be divided into two groups, matched by age, sex, and size. The dogs belong to the Special Operations Unit of the State Police Department, in Porto Alegre, RS, Brazil. Each dog may be fed one meal a day according to his/her weight, and each may be submitted to intense physical activity, such as chasing runaway prisoners or use in police raids or drug sniffing.

The experiment may be divided into a 7-day pre-experimental period and a 30-day experimental period. During the 7-day pre-experimental period, all 20 dogs may be fed Diet A to allow adaptation to the new diet. During the 30-day experimental period, 10 dogs may be fed Diet A and 10 dogs may be fed Diet B. Performance parameters (feed intake, weight gain, body condition, hair coat, stool consistency) and vaccine immune response (vaccine titers) may be evaluated.

Humoral immunity. On day 10 of the experimental period, dogs may receive an immunogen (sheep red blood cells) by subcutaneous injection. On days 0 to 10 after the vaccine administration, 5 ml venous blood samples may be collected in vials without anti-clotting agents. Samples may be centrifuged, and the serum may be separated for counting antibodies against the immunogen.

Qualitative hair analysis. Hair coat may be analyzed before the pre-experimental period begins, and again on days 0, 10, and 30 of the experimental period. A double-blind method may be used to determine a score for the parameters described below. The score accesses both the skin and the hair. The scale of may range from 0 and 3, i.e., with 0=null, 1=low score, 2=average score, and 3=high score. The following parameters may be evaluated: itching, alopecia, erythema, pustules, papules, oily hairs, crusts and scaling, hair loss, shine, and texture at the following sites: face, snout, ventral part of the abdomen, axilae, lateral trunk, fore limbs, hind limbs, lower back, hips, and tail.

Quantitative hair analysis. A hair sample may be collected from the chest area on days 0 and 30 of the experimental period in order to estimate Zn, Cu, and Mn concentration in the hair. For this procedure, an area of 5 cm×5 cm of hair may be trimmed on day 1 of the pre-experimental period. On day 1 of the experimental period, the hair that grew on this site may be removed again, and the hair sample will be frozen for Zn testing. The same procedure may be repeated on day 30.

Serum minerals. A blood sample may be collected with an anti-clotting agent on day 0 and on day 30 of the experimental period to test for serum minerals, especially Zn. This sample may be frozen for future testing.

What is claimed is:

1. A method for promoting hair growth in a subject, the method comprising administering a hydroxy analog of methionine to the subject.

2. The method of claim 1, wherein the hydroxy analog of methionine is a compound having formula (IV): 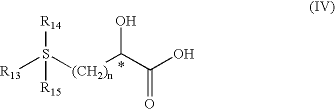
wherein:
* is a chiral carbon;
R₁₃ is methyl or ethyl; and
R₁₄ and R₁₅ are independently oxygen or hydrogen.

3. The method of claim 1, wherein the hydroxy analog of methionine is 2-hydroxy-4(methylthio)butanoic acid.

4. The method of claim 2, wherein the hydroxy analog of methionine is a pharmaceutically acceptable metal salt comprising a compound having formula (IV) together with a metal ion.

5. The method of claim 2, wherein the hydroxyl analog of methionine is a metal chelate comprising a compound having formula (IV) together with a metal ion.

6. The method of claim 5, wherein the metal ion is selected from the group consisting of zinc ions, copper ions, manganese ions, iron ions, chromium ions, nickel ions, cobalt ions, silver ions, selenium ions, magnesium ions and calcium ions.

7. The method of claim 6, wherein the hydroxy analog of methionine is 2-hydroxy-4(methylthio)butanoic acid.

8. The method of claim 1, wherein the hydroxy analog of methionine is a composition formulated for administration to the subject by a route selected from the group consisting of oral, by injection, and topically.

9. The method of claim 8, wherein the composition comprises from about 0.005% to about 20% by weight of the hydroxy analog of methionine.

10. The method of claim 8, wherein the composition further comprises at least one additional agent selected from the group consisting of vitamin, amino acid, coenzyme, antioxidant, minoxidil, finasteride, a peptide metal complex, a penetration enhancement agent, a surface-active agent, an emollient, a propellant, a solvent, a humectant, a thickener, and a powder.

11. The method of claim 8, wherein the composition comprises at least two hydroxy analogs of methionine selected from the group consisting of 2-hydroxy-4(methylthio)butanoic acid, an ester of 2-hydroxy-4(methylthio)butanoic acid, a metal chelate of 2-hydroxy-4(methylthio)butanoic acid, and a pharmaceutically acceptable metal salt of 2-hydroxy-4(methylthio)butanoic acid.

12. The method of claim 11, wherein metal chelate or metal salt comprises metal ions selected from the group consisting of zinc ions, copper ions, manganese ions, iron ions, chromium ions, nickel ions, cobalt ions, silver ions and calcium ions.

13. The method of claim 1, wherein the subject is a human that has an alopecia selected from the group consisting of adrogenetic alopecia, alopecia areata, and secondary alopecia.

14. The method of claim 1, wherein the subject is selected from the group consisting of humans, primates, goats, sheep, pigs, cows, canids, rodents, and equids.

15. A structured fluid delivery system comprising a hydroxy analog of methionine.

16. The structured fluid delivery system of claim 15, wherein the structured fluid delivery system is selected from the group consisting of a liposome, a microemulsion, and a dendrimer.

17. The structured fluid delivery system of claim 15, wherein the structured fluid delivery system comprises a phospholipid bilayer membrane.

18. The structured fluid delivery system of claim 15, wherein the structured fluid delivery system is a liposome having a membrane bilayer comprised of at least one phospholipid selected from the group consisting of phosphatidic acid, phosphatidylserine, phosphatidylinositol, phosphatidylglycerol, diphosphatidylglycerol, phosphatidylcholine, and phosphatidylethanolamine.

19. The structured fluid delivery system of claim 15, wherein the hydroxy analog of methionine is a compound having formula (IV): 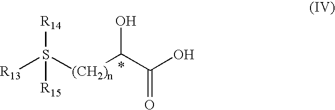
wherein:
* is a chiral carbon;
R₁₃ is methyl or ethyl; and
R₁₄ and R₁₅ are independently oxygen or hydrogen.

20. The structured fluid delivery system of claim 19, wherein the hydroxy analog of methionine is a pharmaceutically acceptable metal salt comprising a compound having formula (IV) together with a metal ion.

21. The structured fluid delivery system of claim 19, wherein the hydroxyl analog of methionine is a metal chelate comprising a compound having formula (IV) together with a metal ion.

22. The structured fluid delivery system of claim 21, wherein the metal ion is selected from the group consisting of zinc ions, copper ions, manganese ions, iron ions, chromium ions, nickel ions, cobalt ions, silver ions, selenium ions, magnesium ions and calcium ions.

23. The structured fluid delivery system of claim 22, wherein the hydroxy analog of methionine is 2-hydroxy-4(methylthio)butanoic acid.

24. The structured fluid delivery system of claim 15, wherein the system comprises from about 0.005% to about 20% by weight of the hydroxy analog of methionine.

25. The structured fluid delivery system of claim 15, wherein the system comprises at least two hydroxy analogs of methionine selected from the group consisting of 2-hydroxy-4(methylthio)butanoic acid, an ester of 2-hydroxy-4(methylthio)butanoic acid, a metal chelate of 2-hydroxy-4(methylthio)butanoic acid, and a pharmaceutically acceptable metal salt of 2-hydroxy-4(methylthio)butanoic acid.

26. A method for selectively delivering a hydroxy analog of methionine to a hair follicle or a skin cell of a subject, the method comprising topically applying to the skin of the subject a composition having a structured fluid delivery system containing a hydroxy analog of methionine, the structured fluid delivery system being capable of selectively delivering the hydroxy analog of methionine to a hair follicle or skin cell disposed within the subject's skin.

27. The method of claim 26, wherein the composition further comprises at least one additional agent selected from the group consisting of vitamin, amino acid, coenzyme, antioxidant, minoxidil, finasteride, a peptide metal complex, a penetration enhancement agent, a surface-active agent, an emollient, a propellant, a solvent, a humectant, a thickener, and a powder.

28. The method of claim 26, wherein the composition comprises at least two hydroxy analogs of methionine selected from the group consisting of 2-hydroxy-4(methylthio)butanoic acid, an ester of 2-hydroxy-4(methylthio)butanoic acid, a metal chelate of 2-hydroxy-4(methylthio)butanoic acid, and a pharmaceutically acceptable metal salt of 2-hydroxy-4(methylthio)butanoic acid.

29. The method of claim 26, wherein the subject is human and has alopecia.
